(19) United States
(12) Patent Application Publication
(10) Pub. No.: US 2024/0216162 A1
(43) Pub. Date: Jul. 4, 2024

(54) Joint Support for a Joint, in Particular for a Knee Joint or Elbow Joint

(71) Applicant: Mario Rumrich, Chemnitz (DE)

(72) Inventor: Mario Rumrich, Chemnitz (DE)

(21) Appl. No.: 18/401,370

(22) Filed: Dec. 30, 2023

(30) Foreign Application Priority Data
Jan. 3, 2023 (DE) 102023100100.0

(51) Int. Cl. A61F 5/01

(52) U.S. Cl. CPC A61F 5/0109; A61F 5/0118

(57) ABSTRACT

A joint support (1) for a joint region of a body part, in particular for a knee joint or elbow joint, having a sleeve-shaped base body (2) which can be stretched in multiple axes, characterized in that the joint support (1) has at least one tension strap (4) which runs over the joint and, in a flexed state of the body part, generates a tensile force (FZ) which promotes a return movement of the body part into a non-flexed position and/or in that the joint support (1) has at least one spring element in the form of a leaf spring (5) on the rear side of the joint, which spring element is tensioned in a flexed state of the body part and provides a restoring force (FR) which promotes a return movement of the body part into a non-flexed position.

### CROSS REFERENCE TO RELATED APPLICATIONS

This application claims the priority of DE 102023100100.0 filed on 2023 Jan. 3; this application is incorporated by reference herein in its entirety.

### BACKGROUND

The invention relates to a joint support for a joint, in particular a knee joint or elbow joint, according to the preamble of the first and second claim.

A dynamic kneecap support is known from the publication EP 10398 B1,
which has an elastic sleeve. The sleeve is provided with an opening over the kneecap and a support pad on the side of the knee. From the support pad, a plurality of straps can be provided around the leg and the sleeve is provided with an opening over the kneecap and a kneecap support pad positioned on the sleeve to provide lateral support for the kneecap during flexion and movement of the knee when the support is in use, the support pad being on the outside of the leg. The support pad has a pair of support arm elements which, in their operative positions, extend from the top and bottom of the stiffening pad, respectively, above and below the kneecap and around the inside of the knee, and which exert medial pressure on the kneecap throughout the stiffening pad. A third arm element, which extends from the support pad on its central axis away from the kneecap and around the outside of the knee and exerts a counterforce on it, and a support pad to hold it in position relative to the kneecap are provided. The third arm element extends from the support pad at its central axis away from the kneecap and around the outside of the knee and exerts a counterforce on the support pad to hold it in position relative to the patella.

U.S. Pat. No. 4,370,978 A describes a knee orthosis for stabilizing a kneecap over the normal range of flexion and movement of the knee, which also has a sleeve element. Attached to it are a first and a second strap for wrapping around above and below the knee; the sleeve element is connected to the first and second strap. Here too, the knee orthosis is provided with a recess in the area of the kneecap.

DE 36 87 154 T2 discloses a support for a knee joint consisting of an essentially trapezoidal sheathing made of textile material, which can be stretched in two directions. Two sloping sides are provided in the area of the knee with semi-elliptical recesses through which the kneecap is also released.

WO 01/56498 A2 also describes a body joint support that leaves the knee free and has crossing straps above and below the knee.

Another orthopedic support is known from DE 3888538 T2 and is an orthopedic support for supporting the knee, elbow, wrist or similar, wherein the joint is completely covered by it and the material is sewn in such a way that an open tubular support is formed.

The upper side of the kneecap is not supported with these solutions, which means that there is a risk of the kneecap moving out of its natural position during very high loads, such as squats with very heavy weights.

All of the above solutions are not suitable for use in weight training under extremely heavy loads.

Despite the use of joint supports or bandages in the knee or elbow area, serious joint injuries and even bone fractures can occur in powerlifting during squats or bench presses, for example.

### SUMMARY

The invention relates to a joint support (1) for a joint region of a body part, in particular for a knee joint or elbow joint, having a sleeve-shaped base body (2) which can be stretched in multiple axes, characterized in that the joint support (1) has at least one tension strap (4) which runs over the joint and, in a flexed state of the body part, generates a tensile force (FZ) which promotes a return movement of the body part into a non-flexed position and/or in that the joint support (1) has at least one spring element in the form of a leaf spring (5) on the rear side of the joint, which spring element is tensioned in a flexed state of the body part and provides a restoring force (FR) which promotes a return movement of the body part into a non-flexed position. (FIG. 1)

### DETAILED DESCRIPTION

The object of the invention is to develop a joint support for a joint, in particular a knee joint or elbow joint, which reliably supports the joint even when an athlete is doing weight training with heavy weights and which supports the athlete when moving from a flexed movement to an extended movement.

This problem is solved with the features of the first and second claim.

Advantageous embodiments result from the subclaims.

The joint support for a joint region of a body part, in particular for a knee joint or elbow joint, has a sleeve-shaped base body which is multi-axially extensible and, according to the invention, is provided with at least one tension strap which runs over the joint and supports the joint and, in a flexed state of the body part, generates a tensile force which promotes a return movement of the body part into a non-flexed position.

Furthermore, the joint support for a joint region of a body part, in particular for a knee joint or elbow joint, can be equipped with a sleeve-shaped base body that can be stretched in multiple axes, wherein the joint support has at least one spring element on the rear side of the joint, which supports and tensions the joint in a flexed state of the body part and provides a restoring force which promotes a return movement of the body part into a non-flexed position.

The two aforementioned variants can be combined with each other, so that on the one hand at least one tension strap runs over the joint and a leaf spring is arranged on the rear.

Both the tension strap and the leaf spring, or a combination of the two, support and protect the joint when it has to cope with heavy weights, such as when squatting with weights or bench pressing.

The risk of injury to the joint capsule, slippage of the kneecap or a cruciate ligament rupture or a tendon rupture or even a bone fracture due to extreme overloading is reduced.

Preferably, the joint support has at least two stretchable tension straps that cross in the joint region.

Training trials have shown that the joint, particularly in the area of the kneecap or elbow, is very well supported under high loads and that the return movement from the flexed to the extended position is very well supported.

The base body is tubular in shape and has a larger diameter at a first end than at a second end. The first end is positioned at the top and the second end at the bottom after application to the corresponding part of the body. When applied to a leg, the first end is located in the area of the thigh and the second end in the area of the calf. When applied to an arm, the first end is located in the area of the upper arm and the second end in the area of the forearm.

It is necessary to ensure that the tension strap(s) run over the joint. If present, the leaf spring must be positioned opposite the tension straps in the crook of the arm or the crook of the leg.

Preferably, the spacing between the tension straps at the first end of the base body is greater than at the second end of the base body, wherein advantageously the tension straps at the first and second ends of the base body are firmly connected to it.

A wrap-around strap is also provided on the base body, which is preferably fastened between the base body and at least one tension strap. In particular, the wrap-around strap is fastened, preferably sewn, below the first end of the base body between an outer edge of a tension strap and the base body.

Furthermore, the wrap-around strap advantageously has a Velcro surface at its free end. When the joint support is in place, the wrap-around strap is wrapped once or several times around the joint support and/or the body part, providing additional support for the joint. The wrap-around strap is then fixed in place with its Velcro surface when applied. In particular, the wrap-around strap is wrapped around the base body under tension.

The wrap-around strap and the tension straps are preferably sewn to the base body. However, the tension straps are not connected to the base body, at least over the joint region.

The tension straps can be unconnected in their crossing region or at least partially connected in their crossing region.

The spring element is preferably arranged between two layers of the base body. A pocket-like insert can also be provided in the base body for this purpose. It is then possible to replace the spring element.

The solution according to the invention thus creates a joint support which, for example, when the leg is bent to perform squats with weight, acts like a spring through the tension strap, which is tensioned during bending. During the return movement to the standing position, the tension strap wants to return to its nontensioned position and supports this return movement to the unbent position of the leg or legs with its tensile force, as a joint support is attached to each leg. Alternatively or in addition to the tension strap, the leaf spring can be arranged on the base body. The leg or arm is then flexed against the spring force of the leaf spring. The leaf spring supports the joint and reinforces the effect of the base body, which is positioned around the joint.

The base body, which is tubular in shape, is dimensioned in such a way that it expands radially when applied and then rests against the body part under tension.

### BRIEF DESCRIPTION OF THE DRAWINGS

The invention is explained in more detail below with reference to exemplary embodiments and associated drawings, wherein:

FIG. 1 shows a front view of the joint support,

FIG. 2 shows an illustration of a joint support applied to a knee,

FIG. 3 shows an illustration of a joint support in which two X-shaped tension straps lead from a small distance above the knee down to the back of the calf,

FIG. 4 shows joint supports applied with bent legs,

FIG. 5 shows a rear view of a joint support which has an integrated spring element in the form of a leaf spring in the area of the knee bend,

FIG. 6 shows a section A-A according to FIG. 5,

FIG. 7 shows a flexed leaf spring, e.g. during knee flexion.

### DETAILED DESCRIPTION OF THE PREFERRED EMBODIMENTS

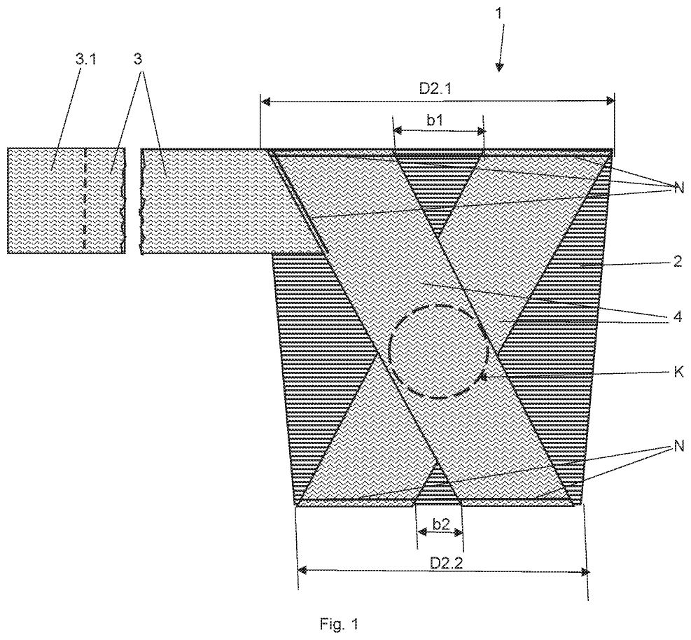

FIG. 1 shows the front view of a joint support 1, which is designed for a knee joint. The joint support 1 has a sleeve-shaped base body 2, which can be stretched in multiple axes. An elastic wrap-around strap 3 is attached to the upper area (first end) of the base body 2, which can be wrapped around the base body 2 and the body part (not shown) and at the end of which a Velcro area 3.1 is provided.

The joint support 1 has two stretchable tension straps 4 that cross in the area of the knee K (indicated by dashed lines) and run over the knee joint or knee K.

The base body 2 is tubular in shape and has a larger diameter D2.1 at a first end, in this case the upper end, and a smaller diameter D2.2 at its second end, in this case the lower end.

The distance b1 between the tension straps 4 at the first end of the base body 2 is greater than the distance b2 at the second end of the base body 2.

The two tension straps 4 are sewn to the base body 2 at their upper and lower ends with a seam N. The tension strap 3 is also sewn here with a seam N between the base body 2 and the tension strap 4.

In the area above the knee K, the tension straps 4 are not connected to each other and run freely over the base body 2.

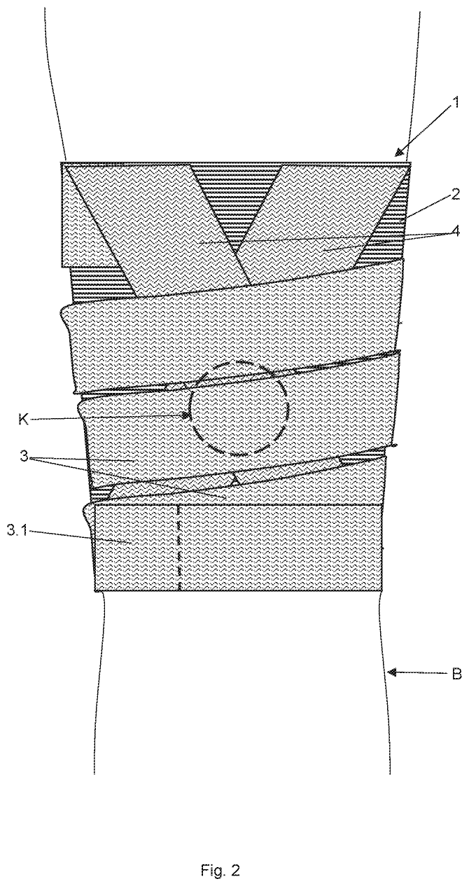

The joint support 1, which is attached to a leg B, is shown in FIG. 2.

The joint support 1 was pulled over the knee K so that the crossing region of the tension straps 4 is located above the knee K. The base body 2 is designed in such a way that it can be stretched in multiple axes and is widened when it is pulled over the knee and then rests against the leg B under tension. The wrap-around strap 3 was wrapped around the base body 2 several times under tension and attached to the wrap-around strap or the base body 2 with the Velcro area 3.1. The tension acting on the wrap-around strap can be generated individually during wrapping.

The fact that the base body 2 is already in contact under tension and is additionally wrapped with the wrap-around strap 3 under tension means that the joint region is already highly stabilized.

If the knee is now flexed, the two crossing tension straps 4 are subjected to tension and pressed against the knee in the area of the kneecap, which results in further stabilization in the knee area with very high weights.

The tension straps also have a supportive effect in order to return the joint to a stretched position.

The invention has a similar effect in the elbow joint region.

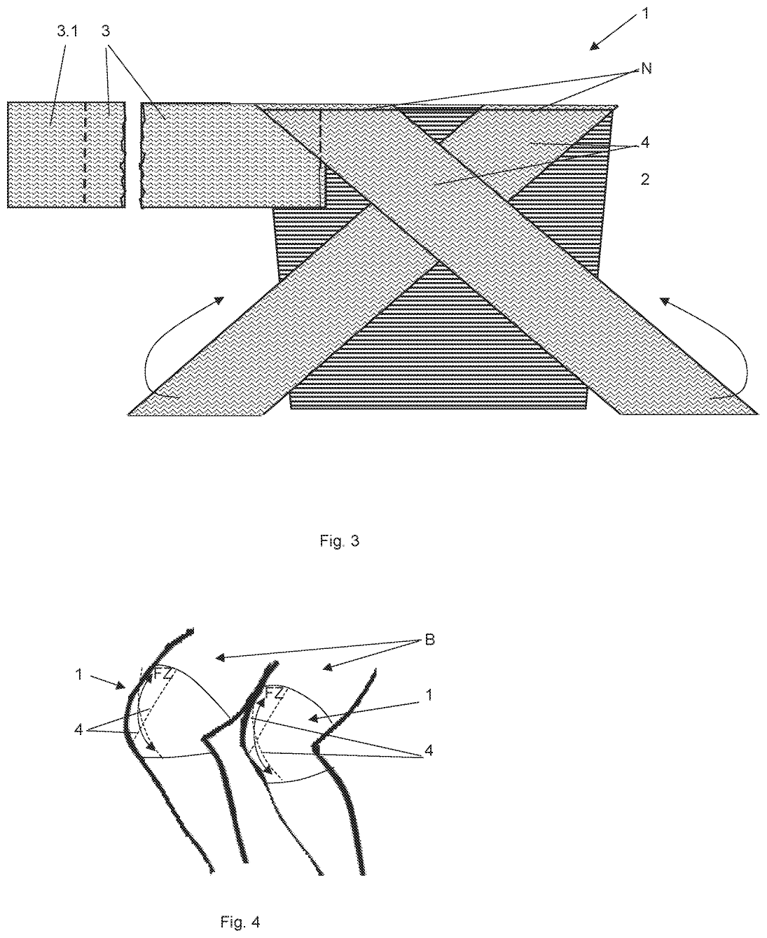

FIG. 3 shows a variant of the joint support 1 in which the crossing tension straps 4 extend on their underside to the back of the base body 2, where they can be attached using Velcro, for example. The tension straps 4 are sewn to the upper side by means of a seam N. The individual fastening of the stretchable tension straps 4 by means of Velcro fastening means that their tension can be individually adjusted.

A wrap-around strap 3 is also provided here, which is stretchable and can be fixed with a Velcro area 3.1 after being wrapped around several times.

FIG. 4 shows that during squats, the joint supports 1 positioned in the area of the knees are “tensioned” with their tension straps 4 (indicated by dashed lines).

As a result, the tension straps 4 strive to return to their nontensioned starting position and promote the return movement of the legs B with a tensile force FZ into an extended position.

Especially with very high weights, as is common in strength sports such as powerlifting, this has a positive effect on the movement sequence and supports the joints, in this case the knee joints.

Due to the very high loads placed on the knees, conventional knee supports can even cause the athlete to break their knees, the kneecap to pop out or the joint capsule or cruciate ligaments to tear.

This is prevented with the joint support according to the invention, as the crossing tension straps extend over the kneecap to stabilize it.

The base body applied under tension and the wrapping with the wrap-around strap has an additional stabilizing effect on the entire knee area.

In addition to the tension straps or as an alternative to them, the joint support 1 can have at least one spring element 5 on the back of the joint, in the knee joint in the area of the knee bend or in the elbow joint in the area of the arm bend, which is tensioned when the body part moves from an extended to a flexed state and provides a restoring force FR, which promotes a return movement of the body part to a non-flexed position.

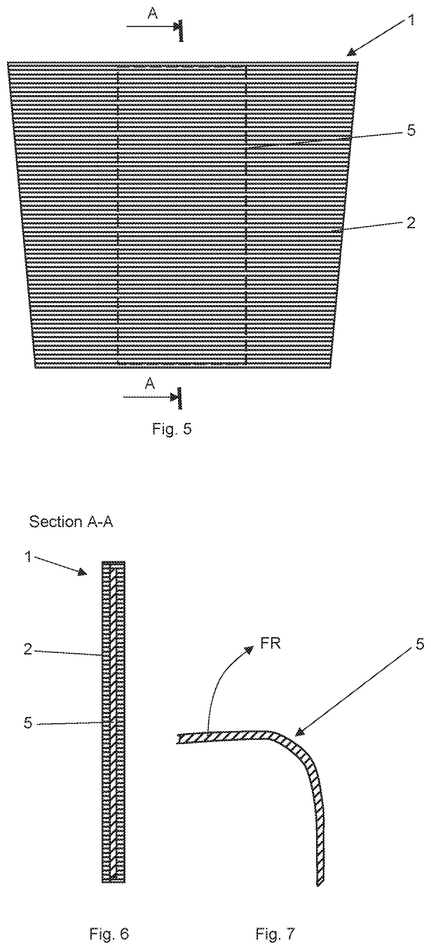

An exemplary embodiment is shown in FIG. 5. The joint support 1 has an essentially rectangular leaf spring 5 on its rear side, which is incorporated into the base body 2 and is preferably located between two layers or in a pocket of the base body 2.

It may also be provided in this variant that a wrap-around strap not shown here is used and that one or more tension straps 4 run over the knee on the opposite side, as shown for example in FIGS. 1 to 4.

FIG. 6 shows the longitudinal section A-A as shown in FIG. 5. It can be seen that the leaf spring 5 is encased in the material of the base body 2.

FIG. 7 shows the individual representation of the leaf spring 5 in a bent state, e.g. when squatting.

A restoring force FR of the leaf spring 5 has a favorable effect in order to move from the flexed position to the extended position.

The leaf spring 5 is preferably made of carbon and can be adapted to the size of the body part and the weight to be supported. It is possible to arrange the leaf spring 5 interchangeably on the base body 2.

In terms of its basic structure and cross-section, the leaf spring 5 is similar to a carbon spring base, such as is used as a running prosthesis for athletes.

Similar to the joint supports 1 for the knees described above, the joint supports 1 can also be used for the elbow joints. The tension straps 4 then preferably run crossed over the elbow.

If a leaf spring 5 is used, it is preferably integrated into the base body 2 or can be inserted and is located in the area of the crook of the arm.

The joint support according to the invention reliably prevents injuries or overloading during competitive sports, especially during exercises with high weights.

### LIST OF REFERENCE NUMERALS

1 Joint support
2 Base body
3 Wrap-around strap
3.1 Velcro area
4 Tension straps
5 Leaf spring
B Legs
N Seam
FR Restoring force
FZ Tensile force

1. Joint support (1) for a joint region of a body part, in particular for a knee joint or elbow joint, having a sleeve-shaped base body (2) which can be stretched in multiple axes, characterized in that the joint support (1) has at least one tension strap (4) which runs over the joint and, in a flexed state of the body part, generates a tensile force (FZ) which promotes a return movement of the body part into a non-flexed position.

2. Joint support (1) for a joint region of a body part, in particular for a knee joint or elbow joint, having a sleeve-shaped base body (2) which can be stretched in multiple axes, characterized in that the joint support (1) has at least one spring element in the form of a leaf spring (5) on the rear side of the joint, which spring element is tensioned in a flexed state of the body part and provides a restoring force (FR) which promotes a return movement of the body part into a non-flexed position.

3. Joint support according to claim 1, characterized in that the joint support (1) has at least two stretchable tension straps (4) crossing in the joint region.

4. Joint support according to claim 1, characterized in that the base body (2) is tubular in shape and has a larger diameter (D2.1) at a first end than at a second end.

5. Joint support according to claim 1, characterized in that the spacing (b1) of the tension straps (4) at the first end of the base body (2) is greater than the spacing (b2) at the second end of the base body (2).

6. Joint support according to claim 1, characterized in that the tension straps (4) at the first and second ends of the base body (2) are firmly connected thereto.

7. Joint support according to claim 1, characterized in that the wrap-around strap (3) is fastened between the base body (2) and at least one tension strap (4).

8. Joint support according to claim 1, characterized in that the wrap-around strap (3) is fastened below the first end of the base body (2) between an outer edge of a tension strap (4) and the base body (2).

9. Joint support according to claim 1, characterized in that the wrap-around strap (3) has a Velcro surface (3.1) at its free end and, in the applied state of the joint support (1), can be wrapped once or several times around the joint support (1) and/or around the body part, and in that the wrap-around strap (3) can be fixed in the applied state by means of its Velcro surface (3.1).

10. Joint support according to claim 1, characterized in that the wrap-around strap (3) and the tension straps (4) are sewn to the base body (2).

11. Joint support according to claim 1, characterized in that the tension straps (4) are not connected to the base body (2) at least over the joint region.

12. Joint support according to claim 1, characterized in that the tension straps (4) are not connected to one another in their crossing region or in that the tension straps (4) are connected to one another at least in certain regions in their crossing region.

13. Joint support according to claim 1, characterized in that the spring element in the form of the leaf spring (5) is arranged between two layers of the base body (2).
